(19) United States
(12) Patent Application Publication
(10) Pub. No.: US 2010/0331997 A1
(43) Pub. Date: Dec. 30, 2010

(54) IMPLANT FOR INTRODUCTION INTO AN ALVEOLAR SPACE

(76) Inventors: Karl-Heinz Sorg, Neustadt/Donau (DE); Claus Martin Hiltner, Nurnberg (DE)

Correspondence Address: MCGLEW & TUTTLE, PC, P.O. BOX 9227, SCARBOROUGH STATION, SCARBOROUGH, NY, 10510-9227, US

(21) Appl. No.: 12/918,430

(22) PCT Filed: Feb. 9, 2009
(86) PCT No.: PCT/EP09/00876

§ 371 Date: Aug. 19, 2010

(30) Foreign Application Priority Data
Feb. 23, 2008 (DE) 10 2008 010 893.6

(51) Int. Cl. A61F 2/28

(52) U.S. Cl. 623/23.57

(57) ABSTRACT

The implant is intended for introduction into an alveolar space. It is configured as an absorbable composite body made of at least a first and a second part body. The first part body is configured as a cone, truncated cone or cylinder having a body base face. The second part body is configured as a cover membrane with a membrane face, which is larger than the body base face. The cover membrane is placed on the body base face, so it projects laterally everywhere over the body base face. The first and second part body are rigidly connected to one another in the region of the mutually adjacent body base face and membrane face.

### CROSS REFERENCE TO RELATED APPLICATIONS

This application is a United States National Phase application of International Application PCT/EP2009/000876 and claims the benefit of priority under 35 U.S.C. §119 of German patent application DE 10 2008 010 893.6 filed Feb. 23, 2008, the entire contents of which are incorporated herein by reference.

### FIELD OF THE INVENTION

The invention relates to an implant for introduction into an alveolar space.

### BACKGROUND OF THE INVENTION

After a tooth extraction, various treatment methods are known in dental medicine. In a first approach, healing of the gingival sulcus (=alveolus) firstly takes place in order to thereafter start again with a dental prosthesis treatment for a tooth implantation, for example. In this case, an undesired shrinkage of bone substance may occur during the healing time. The cause of this is, for example an inflammation in the alveolus, which increases the fibrinolytic activity of the blood, which is why no stable blood clot (=coagulum) can form. There is a disturbed wound healing and a loss of bone tissue. As a result of this, the dental prosthesis can either no longer by optimally anchored or a complex regeneration treatment of the jaw bone even has to be interposed.

In another approach, an implant is inserted directly after the tooth extraction. An example of an implant of this type is, for example, described in DE 196 30 034 A1. It contains a hard titanium core, which is inter alia provided with a collagen-containing coating and is used as the basis for a tooth crown after a growing in phase. With these directly inserted implants, problems may occur during the growing in phase, for example because of inflammations in the alveolus.

### SUMMARY OF THE INVENTION

The object of the invention is to disclose an implant of the type described at the outset, which is easy and reliable to handle and allows good healing of the alveolus.

To achieve this object, an implant for introduction into an alveolar space, which is configured as an absorbable composite body made of at least a first and a second part body, is disclosed, wherein

a) the first part body is configured as a cone, truncated cone or cylinder having a body base face,

b) the second part body is configured as a cover membrane with a membrane face, which is larger than the body base face,

c) the cover membrane is placed on the body base face, so it projects laterally everywhere over the body base face, and

d) the cover membrane is slit once or several times, in particular in an edge region projecting over the body base face, and

e) the first and second part body are rigidly connected to one another in the region of the mutually adjacent body base face and membrane face.

The implant according to the invention allows a complete regeneration of bone and of connective tissue and the epithelium. In particular, it receives the bone substance of the alveolar ridge and allows additional bone tissue to grow into the alveolus, so adequate bone substance is available for later anchoring of the dental prosthesis. After the tooth removal, a stable coagulum forms within the alveolar space when using the implant according to the invention. This coagulum is replaced during the course of wound healing by bone. The implant according to the invention is completely absorbed, in particular, during the wound healing process.

The first part body of the implant according to the invention is very well adapted to the shape of the alveolus because of its (truncated) cone shape. It fills it up very well. Moreover, the first part body has a styptic effect and promotes the formation of a stable coagulum, so a collapse of the alveolar space is reliably prevented. The growing in of bone tissue is therefore encouraged. The implant according to the invention therefore also acts in an osteoconductive manner.

The second part body of the implant according to the invention, on the other hand, prevents connective tissue and the epithelium from growing into the alveolus. This cover membrane covers the alveolus with its edge region projecting over the body base face and thus forms an effective barrier against uncontrolled connective tissue and epithelium proliferation into the alveolus. As bone tissue proliferates substantially more slowly in comparison to connective tissue and the epithelium, without the cover membrane there would be a risk of the connective tissue and epithelium filling up the alveolus space more rapidly and therefore inhibiting the bone growth.

Because of the rigid connection between the two part bodies it is always ensured that the cover membrane is in the correct position in relation to the (truncated) cone and the alveolus and also remains in this position during the healing of the wound. The rigid connection between the two part bodies also prevents errors occurring during insertion, for example the cover membrane being incorrectly positioned and not completely covering the alveolus. The composite body of the implant according to the invention is therefore particularly simple and reliable to handle and shortens the treatment period.

Overall, the implant according to the invention provides a “regenerative space”, because of which the physiological wound healing is encouraged and a loss of bone mass is avoided.

A variant is favorable, in which the first part body consists at least partially of a porous collagen, for example of collagen which is lyophilized, dried or produced by means of felting, preferably of reconstituted collagen of type 1. It is furthermore preferably provided that the collagen of the first part body has a density of 1 to 25 mg/cm³, preferably of 5 to 12 mg/cm³. Collagen with these density values can be produced particularly well. Collagen, as a bioabsorbable material, is very well tolerated and is used to arrest bleeding, for filling bone and tissue defects and for covering wounds. Collagen assists the arrest of bleeding in that thrombocytes clump together on the collagen fibrils and a coagulum forms. Collagen is completely absorbed in the course of wound healing owing to the effect of migrated macrophages and collagenase particular to the body.

According to another advantageous variant, the first part body has an, in particular, solid, cylindrical or conical core made of native, prepared bone material, such as, for example, spongiosa, or a synthetic replacement bone material, such as, for example, tricalcium phosphate, hydroxylapatite or a similar ceramic material. As an alternative to this, the first part body may, however, likewise preferably consist of a mixed material with a collagen fraction and a bone fraction incorporated homogenously distributed in the collagen fraction, the bone fraction being formed by replacement bone material or by comminuted native, prepared bone material, such as, for example, spongiosa. The bone fraction added in the first variant by means of the separate core or homogenously mixed in in the second variant in each case fluctuates, in particular in the range of between 5% and 90% of the volume of the first part body. The two variants encourage bone formation in the alveolar space during the wound healing process.

According to a further favorable configuration, the second part body consists of a compacted collagen with, in particular, a smaller pore size than that of the collagen of the first part body. The compacted collagen, in particular, forms a film, a foil or a compacted sponge. The compacted collagen of the second part body is preferably a compacted lyophilisate made of reconstituted collagen. In contrast to the porous first part body, in which the wound healing and tissue formation are in the foreground, the second part body is used as a barrier against connective tissue and the epithelium growing in. Tissue formation which is especially not desired at this point is prevented by the use of compacted collagen or at least made significantly more difficult. The collagen of the second part body is, in particular, denser by a factor or 0.5 to 5 than that of the first part body.

According to a further preferred configuration, at least one of the two part bodies may consist of a material, which at least contains a bioactive constituent which is wound-healing and/or encourages the bone growth. This bioactive component is preferably a native isolated or biotechnologically obtained protein, namely BMP-2 (=Bone Morphogenic Protein 2). This inter alia also includes TGF beta (TGF=Transforming Growth Factor) or similar, in other words, in particular growth factors in general.

According to another also favorable configuration, at least one of the two part bodies may consist of a material which at least contains one antimicrobial constituent. This antimicrobial constituent contributes to preventing infections and/or fighting them. This is preferably a locally tolerable antiseptic, such as, for example, polyhexanide, oxtenidine, silver ions, iodine derivatives or the like. Likewise, an antibiotically acting substance suitable for local application, such as, for example, gentamicin, metronidazole, vancomycin or the like may be used.

A variant is also favorable, in which the cover membrane is slit once or several times, in particular in an edge region projecting over the base body face. This further simplifies the application to cover the alveolus.

It is also preferably provided that the composite body consists at least partially, in particular the (truncated) cone or the cover membrane, in part or completely, of a gelatin or an oxidized cellulose. These materials are also completely absorbable and well tolerated biologically. The gelatin may, in this case, in particular, also be a collagen-containing material, the collagen of which is more strongly denatured than in the above-mentioned collagen materials (=lyophilized and/or reconstituted collagen of type 1). The oxidized cellulose is another biopolymer. The composite body may also consist of a mixture of one of the previously mentioned collagen materials and the gelatin and/or the oxidized cellulose.

According to further advantageous configurations, the composite body has the particularly favorable geometric dimensions mentioned below, which leads to a very good adaptation to the purpose of use, in other words, insertion into the alveolus and the covering of the alveolus. The overall height of the composite body is, in particular, at least 1.6 cm. Furthermore, the body base face of the first part body has a preferred diameter of 1 cm to 1.5 cm, preferably of 1.3 cm. A cone height of the first part body is, in particular, between 1 cm and 3 cm, preferably between 1.3 cm and 1.7 cm. The second part body preferably has a membrane thickness of between 0.05 cm and 0.5 cm and a membrane diameter, which is larger by 0.2 cm to 2 cm than a diameter of the body base face and which is, in particular, at least 1.4 cm.

According to a further advantageous configuration, the cover membrane comprises an approximately circular center portion, on the outer periphery of which at least one elongate, laterally projecting membrane wing is attached in one piece. In particular, two membrane wings of this type are provided, which oppose one another. They are used to cover vestibular and palatinal bone defects.

The various features of novelty which characterize the invention are pointed out with particularity in the claims annexed to and forming a part of this disclosure. For a better understanding of the invention, its operating advantages and specific objects attained by its uses, reference is made to the accompanying drawings and descriptive matter in which preferred embodiments of the invention are illustrated.

### BRIEF DESCRIPTION OF THE DRAWINGS

In the drawings:

FIG. 1 shows an embodiment of an absorbable implant as a composite body made of a truncated cone and a cover membrane in an exploded view;

FIG. 2 shows the implant according to FIG. 1 in a plan view from above onto the cover membrane;

FIG. 3 shows a tooth seated in an alveolus in a cross sectional view;

FIG. 4 shows an embodiment of an absorbable implant consisting of a truncated cone and a cover membrane, which is inserted, after an extraction of the tooth, into the alveolus according to FIG. 3;

FIG. 5 to 7 show a further embodiment of an absorbable implant consisting of a truncated cone and a laterally extended cover membrane in a perspective view, in a view from below and in a side view; and

FIG. 8 shows the implant according to FIG. 5 to 7 in an inserted state.

### DESCRIPTION OF THE PREFERRED EMBODIMENTS

Parts which correspond with one another are provided with the same reference numerals in FIG. 1 to 8.

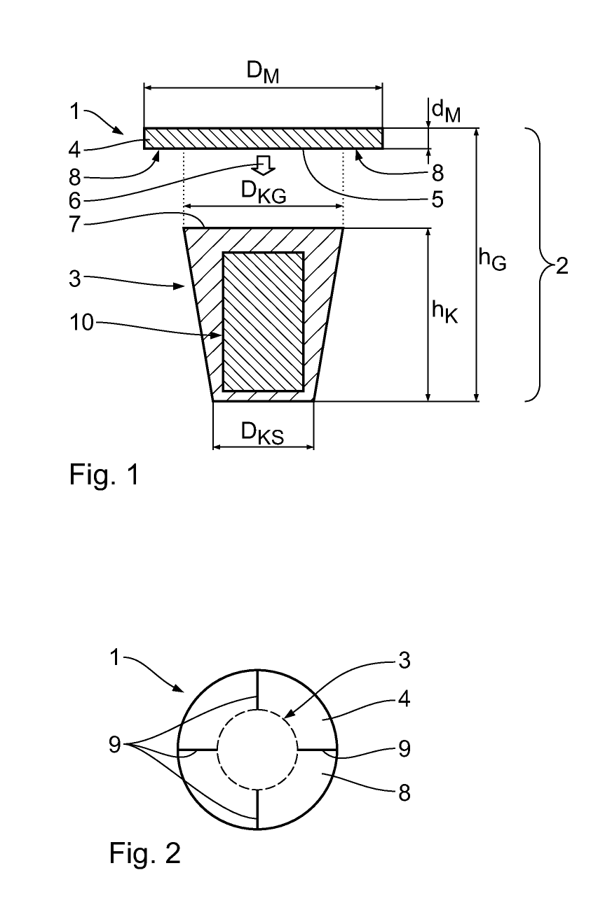

FIG. 1 shows an embodiment of an absorbable implant 1, which is configured as a composite body 2 made of two part bodies. The first part body is a truncated cone 3 and the second part body is a substantially round cover membrane 4. The truncated cone 3 and the cover membrane 4 are rigidly connected to one another in the absorbable implant 1. The cover membrane 4 with a lower membrane face 5, as indicated in FIG. 1 by the arrow 6, is placed approximately concentrically on a body base face of the truncated cone 3 configured as an upper base cone face 7. The membrane face 5 is larger than the base cone face 7. It projects, in an end region 8, beyond the base cone face 7. The cover membrane 4 is slit in the end region 8. In order to allow simpler application, in the embodiment shown (see plan view according to FIG. 2), a total of four slits 9 arranged uniformly distributed about the periphery and extending in the radial direction are provided.

In the embodiment, the base cone face 7 of the truncated cone 3 has a diameter D_{KG} of about 1.3 cm. A diameter D_{KS} at the narrow end of the truncated cone 3 is about 0.9 cm. Its height h_{K} is about 1.5 cm. The cover membrane 4 has a membrane diameter D_{M} of about 2 cm and a membrane thickness d_{M} of about 0.1 cm, so an overall height h_{G} of the composite body 2 is about 1.6 cm.

In the region, in which the membrane face 5 and the base cone face 7 adjoin one another, the truncated cone 3 and the cover membrane 4 are rigidly connected to one another by means of the adhesive force of the collagen-containing materials, from which the truncated cone 3 and the cover membrane 4 are produced in each case. The two parts 3 and 4 of the composite body 2 are joined together as long as the collagen-containing material has not yet dried in at least one of the two parts, so the natural adhesive force effect of the collagen-containing material is used to produce the rigid connection. This is favorable as, in this manner, no separate adhesive or connecting means are required.

The truncated cone 3, in the embodiment shown in FIG. 1, consists of a porous collagen lyophilisate with a density of 7 mg/cm³. Enclosed therein is optionally a cylindrical or (truncated) cone-shaped core 10, also shown in FIG. 1, made of a bone material, for example of native, prepared spongiosa material or of a synthetically produced material such as tricalcium phosphate or hydroxylapatite.

The cover membrane 4, in the embodiment shown in FIG. 1, consists of a compacted lyophilisate made of reconstituted collagen. The collagen material of the cover membrane 4 has a smaller pore size than that of the truncated cone 3. The cover membrane 4 is, for example, configured as a foil or a compacted sponge made of the collagen mentioned. Its density is higher by a factor of about 2 than that of the truncated cone 3.

The implant 1 is produced, for example, as follows. Firstly, the cover membrane 4 is lyophilized, pressed and cut to size. A liquid collagen suspension is then introduced into a truncated cone shape to form the truncated cone 3. The cover membrane 4 is applied to this collagen suspension, as long as the latter is still in a liquid state. By means of a concluding lyophilisation step, the composite body 2 with the porous collagen truncated cone 3 and the collagen cover membrane 4 is produced as a rigidly connected unit.

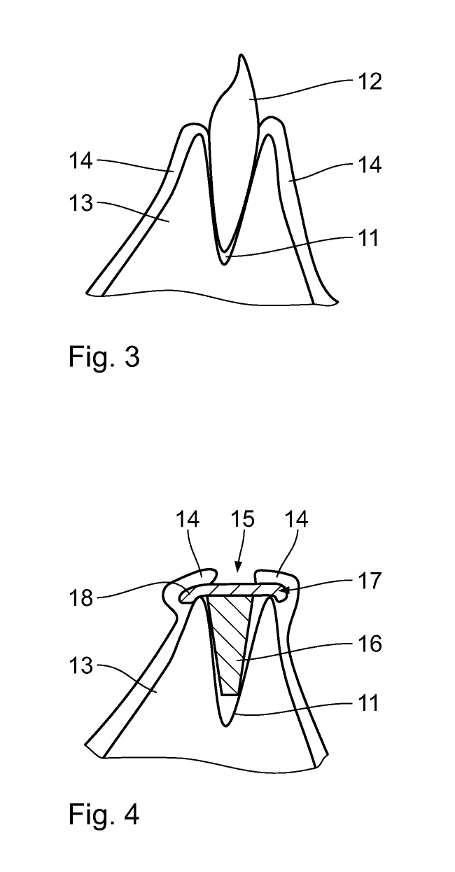

FIG. 3 shows a tooth 12 seated in an alveolus 11. The alveolus 11 is formed by the jaw bone 13, which is covered on its outside by connective tissue and the epithelium 14.

After the extraction of the tooth 12, the alveolus 11 is immediately fitted with an absorbable implant 15, which is structured substantially like the implant 1 according to FIGS. 1 and 2 (see FIG. 4). In contrast to the implant 1, a truncated cone 16 of the implant 15 does not contain a core made of (replacement) bone material. Otherwise, the implant 15, precisely like the implant 1, has a cover membrane 17 also rigidly connected to the truncated cone 16.

Because of its conical contour adapted to the alveolar space, the truncated cone 16 fills up the alveolus 11 in the entire area very well, so a stable coagulum promoting wound healing can be formed in the entire alveolus 11. The truncated cone 16 is plastically deformable and therefore fits the alveolus 11 very well. A stabilization of the coagulum in the entire alveolus 11 is thus achieved. The truncated cone 16 acts osteoconductively, i.e. it facilitates the growing of new bone tissue into the alveolus 11.

The truncated cone 16 is also kept in the correct position, apart from by its shape, by the cover membrane 17, which rests with its laterally projecting edge region 18 on the jaw bone 13. Because of the cover membrane 17, a very precise vertical and horizontal positioning of the implant 15 is possible as a whole. The application is made simpler and becomes more reliable. Because of the fixed combination, there can be no relative displacement between the cover membrane 17 and the truncated cone 16. Moreover, the cover membrane 17 fulfills a function protecting the alveolar space, so the connective tissue and epithelium 14 are prevented from undesirably growing into the alveolus 11. As bone tissue, in contrast to connective tissue and epithelium 14 proliferates substantially more slowly, the connective tissue and epithelium 14, without the barrier formed by the cover membrane 17, would more quickly fill up the alveolar space and therefore inhibit the bone growth.

The implants 1 and 15 are absorbable and, in particular, also have a styptic effect. Inserted into the alveolus 11 (gingival sulcus), after a tooth extraction, they bring about an arrest of bleeding and advantageously prevent a collapse of the alveolus 11. With the continuing inflammation-free healing process, a bone formation within the alveolus 11 is also even promoted (=osteoconductive effect). The implants 1 and 15 thus provide a “regenerative space”, by means of which the physiological wound healing is encouraged and a loss of bone mass is avoided. The bone mass thus obtained or additionally built up of the alveolus 11 may then be used as a basis for an implantation provided later of a dental prosthesis.

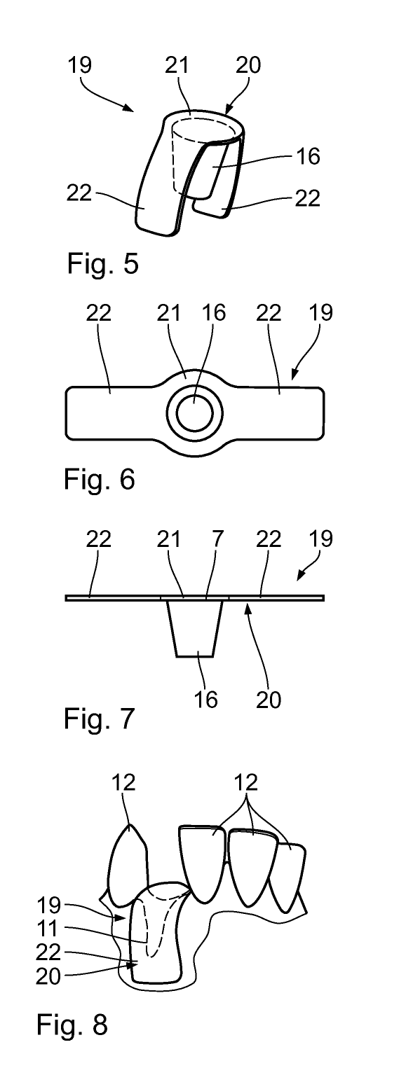

A further embodiment of an absorbable implant 19 is shown in FIG. 5 to 7. It comprises a similar plastically deformable truncated cone 16 like the implant 15 according to FIG. 4. A cover membrane 20 consisting of collagen is also rigidly connected to the truncated cone 16 in the implant 19. The cover membrane 20 is designed slightly differently to the cover membrane 4 or 17 according to FIGS. 1 and 2 or 4. It also has an approximately circular center portion 21, which is in turn larger than the base cone face 7 of the truncated cone 16 (cf the view from below according to FIG. 6), but two lateral membrane wings 22 are in each case additionally formed on in one piece on the center portion 21. The two elongate membrane wings 22 oppose one another. They are thus attached to the center portion 21 offset with respect to one another by about 180° in the peripheral direction of the center portion 21. Their extent in the longitudinal direction is in each case preferably approximately just as long as the height hₖ of the truncated cone 16. Optionally their length may, however, also be greater than the height hₖ of the truncated cone 16. The membrane wings 22 are lateral elongations of the center portion 21.

The effect of the membrane wings 22 emerges from the figure of the implant 19 in its inserted state shown in FIG. 8. The membrane wings 22 cover the region of the alveolus 11 in a vestibular and palatinal manner. This is advantageous, in particular, if bone defects exist in this region. The membrane wings 22 then also prevent, at these points, an uncontrolled advance of connective tissue and epithelium, which would otherwise be disadvantageous to a regeneration of the bone tissue.

While specific embodiments of the invention have been described in detail to illustrate the application of the principles of the invention, it will be understood that the invention may be embodied otherwise without departing from such principles.

1. An implant for introduction into an alveolar space, which is configured as an absorbable composite body made of at least a first and a second part body, wherein
the first part body is configured as one of a group of a cone, a truncated cone and a cylinder having a body base face,
the second part body is configured as a cover membrane with a membrane face, which is larger than the body base face,
the cover membrane is placed on the body base face, so it projects laterally everywhere over the body base face, and
the cover membrane is slit one of once and several times in an edge region projecting over the body base face, and
the first and second part body are rigidly connected to one another in the region of the mutually adjacent body base face and membrane face.

2. An implant according to claim 1, wherein the first part body consists at least partially of a porous collagen.

3. An implant according to claim 2, wherein the collagen of the first part body has a density of 1 to 25 mg/cm³.

4. An implant according to claim 1, wherein the first part body has a core of one of the group of native, prepared bone material and synthetic replacement bone material.

5. An implant according to claim 1, wherein the first part body consists of a mixed material with a collagen fraction and a bone fraction incorporated homogenously distributed in the collagen fraction, the bone fraction being formed by one of the group of replacement bone material and comminuted native, prepared bone material.

6. An implant according to claim 1, wherein the second part body consists of a compacted collagen.

7. An implant according to claim 6, wherein the compacted collagen of the second part body is a compacted lyophilisate made of reconstituted collagen.

8. An implant according to claim 1, wherein at least one of the two part bodies consists of a material, which contains at least one bioactive constituent which is one of the group of wound-healing and promotes bone growth.

9. An implant according to claim 1, wherein at least one of the two part bodies consists of a material which contains at least one of the group of antimicrobial and antibiotically acting constituent.

10. An implant according to claim 1, wherein the composite body at least partially consists of one of the group of a gelatin and an oxidized cellulose.

11. An according to claim 1, wherein an overall height of the composite body is at least 1.6 cm.

12. An implant according to claim 1, wherein in the first part body, the body base face has a diameter of 1 cm to 1.5 cm and a cone height is between 1 cm and 3 cm.

13. An implant according to claim 1, wherein the second part body has a membrane thickness of between 0.05 cm and 0.5 cm and a membrane diameter, which is larger by 0.2 cm to 2 cm than a diameter of the body base face.

14. An implant according to claim 1, wherein the cover membrane comprises an approximately circular center portion, on the outer periphery of which at least one elongate, laterally projecting membrane wing is attached in one piece.

15. An implant according to claim 1, wherein the first part body consists at least partially of a reconstituted collagen of type 1.

16. An implant according to claim 3, wherein the collagen of the first part body has a density of 5 to 12 mg/cm³.

17. An implant according to claim 1, wherein the second part body consists of a compacted collagen having a smaller pore size than that of the collagen of the first part body and forming one of the group of a film, a foil and a compacted sponge.

18. An implant according to claim 1, wherein in the first part body, the body base face has a diameter of 1 cm to 1.5 cm and a cone height is between 1.3 cm and 1.7 cm.

19. An implant according to claim 1, wherein in the first part body, the body base face has a diameter of 1.3 cm and a cone height is between 1 cm and 3 cm.

20. An implant according to claim 1, wherein in the first part body, the body base face has a diameter of 1.3 cm and a cone height is between 1.3 cm and 1.7 cm.

21. An implant according to claim 1, wherein the second part body has a membrane thickness of between 0.05 cm and 0.5 cm and a membrane diameter which is at least 1.4 cm.
